(19) United States
(12) Patent Application Publication
(10) Pub. No.: US 2019/0060677 A1
(43) Pub. Date: Feb. 28, 2019

(54) SYSTEM AND METHOD FOR ULTRASONICALLY SENSING AND ABLATING TISSUE

(71) Applicant: VytronUS, Inc., Sunnyvale, CA (US)

(72) Inventors: Hira V. Thapliyal, Los Altos, CA (US); David A. Gallup, Alameda, CA (US); James W. Arenson, Portland, OR (US)

(21) Appl. No.: 16/170,566

(22) Filed: Oct. 25, 2018

Related U.S. Application Data
(63) Continuation of application No. 15/350,590, filed on Nov. 14, 2016, now abandoned.
Application No. 15/350,590 is a continuation of application No. 12/695,857, filed on Jan. 28, 2010, now abandoned.
(60) Provisional application No. 61/148,809, filed on Jan. 30, 2009.

(51) Int. Cl. A61N 7/02; A61B 8/08; A61B 8/00; A61B 8/12; A61B 8/14; A61B 18/00; A61B 90/00; A61N 7/00

(52) U.S. Cl. CPC A61N 7/022; A61B 2018/00577; A61B 8/54; A61B 8/12; A61B 8/14; A61B 8/4483; A61B 8/483; A61B 8/0883; A61B 8/085; A61B 8/543; A61B 2018/00351; A61B 2018/00357; A61B 2018/00375; A61B 2090/3782; A61N 2007/0052; A61B 8/5207

(57) ABSTRACT

Echo-anatomically mapping tissue includes advancing a catheter having an ultrasound transducer toward tissue. A console adjacent the proximal end of the catheter controls catheter movement, and the ultrasound transducer senses tissue. First and second regions of the tissue are ultrasonically sensed while moving the ultrasound transducer along first, and second sensing patterns, respectively. A first 3-dimensional surface map of the first region, and a second 3-dimensional surface map of the second region are generated. The 3-dimensional surface maps are combined to form a combined surface map. Anatomical features may be identified in the first or second sensed regions. The tissue may be ultrasonically ablated while moving the ultrasound transducer along a first ablation path. The first ablation path may form a lesion around the identified anatomical features, and may be selected from a catalog of ablation paths or it may be prescribed by a physician.

### CROSS-REFERENCE

This application is a continuation application of Ser. No. 15/350,590 (Attorney Docket No. 31760-712.301), filed Nov. 14, 2016; which is a continuation application of Ser. No. 12/695,857 (Attorney Docket No. 31760-712.201), filed Jan. 28, 2010; which is a non-provisional of, and claims the benefit of U.S. Provisional Patent Application No. 61/148,809 (Attorney Docket No. 31760-712.101), filed Jan. 30, 2009, to which application we claim priority under 35 U.S.C. § 120; the full disclosures of which are incorporated herein by reference in their entirety.

The present application is also related to the following U.S. patent applications Ser. No. 11/747,862 (Attorney Docket No. 31760-703.201); Ser. No. 11/747,867 (Attorney Docket No. 31760-703.202); Ser. No. 12/480,929 (Attorney Docket No. 31760-704.201); Ser. No. 12/480,256 (Attorney Docket No. 31760-705.201); Ser. No. 12/483,174 (Attorney Docket No. 31760-706.201); Ser. No. 12/482,640 (Attorney Docket No. 31760-707.201); Ser. No. 12/505,326 (Attorney Docket No. 31760-708.201); Ser. No. 12/505,335 (Attorney Docket No. 31760-709.201); Ser. No. 12/620,287 (Attorney Docket No. 31760-711.201); Ser. No. 12/609,759 (Attorney Docket No. 31760-713.201); Ser. No. 12/609,274 (Attorney Docket No. 31760-716.201); Ser. No. 12/609,705 (Attorney Docket No. 31760-718.201); and U.S. Provisional Patent Application No. 61/254,997 (Attorney Docket No. 31760-31760-720.101). The entire contents of each of the above listed patent applications is incorporated herein by reference.

### BACKGROUND OF THE INVENTION

### 1. Field of the Invention

The present application generally relates to systems and methods for creating ablation zones in human tissue. More specifically, the present application relates to the treatment of atrial fibrillation of the heart by using ultrasound energy. While the present application emphasizes treatment of atrial fibrillation, one of skill in the art will appreciate that this it not intended to be limiting, and that the systems and methods disclosed herein may also be used to treat other tissues and conditions, including other arrhythmias like ventricular fibrillation.

The condition of atrial fibrillation is characterized by the abnormal (usually very rapid) beating of the left atrium of the heart which is out of synch with the normal synchronous movement (normal sinus rhythm') of the heart muscle. In normal sinus rhythm, the electrical impulses originate in the sino-atrial node (‘SA node’) which resides in the right atrium. The abnormal beating of the atrial heart muscle is known as ‘fibrillation’ and is caused by electrical impulses originating instead at points other than the SA node, for example, in the pulmonary veins (PV).

There are pharmacological treatments for this condition with varying degree of success. In addition, there are surgical interventions aimed at removing the aberrant electrical pathways from PV to the left atrium (LA') such as the ‘Cox-Maze III Procedure’. This procedure has been shown to be 99% effective but requires special surgical skills and is time consuming. Thus, there has been considerable effort to copy the Cox-Maze procedure using a less invasive percutaneous catheter-based approach. Less invasive treatments have been developed which involve use of some form of energy to ablate (or kill) the tissue surrounding the aberrant focal point where the abnormal signals originate in PV. The most common methodology is the use of radio-frequency (‘RF’) electrical energy to heat the muscle tissue and thereby ablate it. The aberrant electrical impulses are then prevented from traveling from PV to the atrium (achieving the ‘conduction block’) and thus avoiding the fibrillation of the atrial muscle. Other energy sources, such as microwave, laser, and ultrasound have been utilized to achieve the conduction block. In addition, techniques such as cryoablation, administration of ethanol, and the like have also been used. Some of these methods and devices are described below.

There has been considerable effort in developing catheter based systems for the treatment of AF using radiofrequency (RF) energy. One such method includes a catheter having proximal and distal electrodes at the catheter tip. The catheter can be bent in a coil shape, and positioned inside a pulmonary vein. The tissue of the inner wall of the PV is then ablated in an attempt to kill the source of the aberrant heart activity.

Another source used in ablation is microwave energy. One such intraoperative device consists of a probe with a malleable antenna which has the ability to ablate the atrial tissue.

Still another catheter based method utilizes the cryogenic technique where the tissue of the atrium is frozen below a temperature of −60 degrees C. This results in killing of the tissue in the vicinity of the PV thereby eliminating the pathway for the aberrant signals causing the AF. Cryo-based techniques have also been a part of the partial Maze procedures described above. More recently, Dr. Cox and his group have used cryoprobes (cryo-Maze) to duplicate the essentials of the Cox-Maze III procedure.

Other recent approaches for the treatment of AF involve the use of ultrasound energy. The target tissue of the region surrounding the pulmonary vein is heated with ultrasound energy emitted by one or more ultrasound transducers. One such approach includes a catheter distal tip portion equipped with a balloon and containing an ultrasound element. The balloon serves as an anchoring means to secure the tip of the catheter in the pulmonary vein. The balloon portion of the catheter is positioned in the selected pulmonary vein and the balloon is inflated with a fluid which is transparent to ultrasound energy. The transducer emits the ultrasound energy which travels to the target tissue in or near the pulmonary vein and ablates it. The intended therapy is to destroy the electrical conduction path around a pulmonary vein and thereby restore the normal sinus rhythm. The therapy involves the creation of a multiplicity of lesions around individual pulmonary veins as required.

Yet another catheter device using ultrasound energy includes a catheter having a tip with an array of ultrasound elements in a grid pattern for the purpose of creating a three dimensional image of the target tissue. An ablating ultrasound transducer is provided which is in the shape of a ring which encircles the imaging grid. The ablating transducer emits a ring of ultrasound energy at 10 MHz frequency.

In many of the above approaches, the devices and systems involve the ablation of tissue inside a pulmonary vein or of the tissue at the location of the ostium. This may require complex positioning and guiding of the treatment devices to the target site. The ablation is achieved by means of contact between the device and the tissue. Also, many of these systems often require a catheter to be repositioned multiple times within the heart in order to map the atrium or other chamber. Repositioning may require complex manipulation of the catheter and thus this process can be cumbersome.

Other ablation systems may be used to map tissue surfaces. For example, one commercially available system uses a high energy focused ultrasound (HIFU) catheter to capture two-dimensional images of a prostate gland relating to blood flow in the target tissue. The user then manually marks tissue components on the individual 2-dimensional images. Thereafter, the images are formed into a three-dimensional model, and a chosen area is ablated in a pinpoint manner. A table, which maps transducer parameters to expected lesion size, is employed to aid in ablation. During the process, the transducer must be repeatably positioned at the same location in order for the method to be effectively carried out. While promising, this system is not optimized for ablation of cardiac tissue. Therefore, it would also be advantageous to provide an ablation system that can ultrasonically sense and scan the portion of the heart to be ablated, and that can create a 3-dimensional surface map of the tissue surface based on the scanned data.

It would further be advantageous if such systems could identify anatomical features such as pulmonary veins on the surface map, and suggest an ablation path surrounding the anatomical features. It would also be advantageous if such systems could ablate along the suggested ablation path using the same catheter that was used for sensing and scanning. At least some of these objectives will be met by the present invention.

In the cardiac field methods exits for treating cardiac arrhythmias with no discrete target. A description of the heart chamber anatomy, such as the physical dimensions of the chamber, is obtained and an activation map of a patient's heart is created using locatable catheters. A conduction velocity map is derived from the activation map. Then, a refractory period map is acquired. Appropriate values from the conduction velocity map and the refractory period map are used to create a dimension map, which is then analyzed to determine ablation lines or points. This mapping is promising, but it would also be advantageous to provide a single system that ultrasonically ablates and senses the cardiac tissue and generates 3-dimensional tissue map. It would be additionally useful to provide a system that is configured to identify desired anatomical features on the 3-dimensional tissue map. Further, it would be beneficial to provide a catalog of lesion paths to choose from when ablating on a path around one or more desired anatomical features. At least some of these objectives will be met by the present invention.

### 2. Description of Background Art

Patents related to the treatment of atrial fibrillation include, but are not limited to the following: U.S. Pat. Nos. 6,997,925; 6,996,908; 6,966,908; 6,964,660; 6,955,173; 6,954,977; 6,953,460; 6,949,097; 6,929,639; 6,872,205; 6,814,733; 6,780,183; 6,666,858; 6,652,515; 6,635,054; 6,605,084; 6,547,788; 6,514,249; 6,502,576; 6,416,511; 6,383,151; 6,305,378; 6,254,599; 6,245,064; 6,164,283; 6,161,543; 6,117,101; 6,064,902; 6,052,576; 6,024,740; 6,012,457; 5,718,241; 5,405,346; 5,314,466; 5,295,484; 5,246,438; and 4,641,649.

Patent Publications related to the treatment of atrial fibrillation include, but are not limited to International PCT Publication Nos. WO 2005/117734; WO 99/02096; and U.S. Patent Publication Nos. 2007/0219448; 2005/0267453; 2003/0050631; 2003/0050630; and 2002/0087151.

Scientific publications related to the treatment of atrial fibrillation include, but are not limited to: Haissaguerre, M. et al., Spontaneous Initiation of Atrial Fibrillation by Ectopic Beats Originating in the Pulmonary Veins, New England J. Med., Vol. 339:659-666; J. L. Cox et al., The Development of the Maze Procedure for the Treatment of Atrial Fibrillation, Seminars in Thoracic & Cardiovascular Surgery, 2000; 12: 2-14; J. L. Cox et al., Electrophysiologic Basis, Surgical Development, and Clinical Results of the Maze Procedure for Atrial Flutter and Atrial Fibrillation, Advances in Cardiac Surgery, 1995; 6: 1-67; J. L. Cox et al., Modification of the Maze Procedure for Atrial Flutter and Atrial Fibrillation. II, Surgical Technique of the Maze III Procedure, Journal of Thoracic & Cardiovascular Surgery, 1995; 110:485-95; J. L. Cox, N. Ad, T. Palazzo, et al. Current Status of the Maze Procedure for the Treatment of Atrial Fibrillation, Seminars in Thoracic & Cardiovascular Surgery, 2000; 12: 15-19; M. Levinson, Endocardial Microwave Ablation: A New Surgical Approach for Atrial Fibrillation; The Heart Surgery Forum, 2006; Maessen et al., Beating Heart Surgical Treatment of Atrial Fibrillation with Microwave Ablation, Ann Thorac Surg 74: 1160-8, 2002; A. M. Gillinov, E. H. Blackstone and P. M. McCarthy, Atrial Fibrillation: Current Surgical Options and their Assessment, Annals of Thoracic Surgery 2002; 74:2210-7; Sueda T., Nagata H., Orihashi K., et al., Efficacy of a Simple Left Atrial Procedure for Chronic Atrial Fibrillation in Mitral Valve Operations, Ann Thorac Surg 1997; 63:1070-1075; Sueda T., Nagata H., Shikata H., et al.; Simple Left Atrial Procedure for Chronic Atrial Fibrillation Associated with Mitral Valve Disease, Ann Thorac Surg 1996; 62:1796-1800; Nathan H., Eliakim M., The Junction Between the Left Atrium and the Pulmonary Veins, An Anatomic Study of Human Hearts, Circulation 1966; 34:412-422; Cox J. L., Schuessler R. B., Boineau J. P., The Development of the Maze Procedure for the Treatment of Atrial Fibrillation, Semin Thorac Cardiovasc Surg 2000; 12:2-14; and Gentry et al., Integrated Catheter for 3-D Intracardiac Echocardiography and Ultrasound Ablation, IEEE Transactions on Ultrasonics, Ferroelectrics, and Frequency Control, Vol. 51, No. 7, pp 799-807.

### BRIEF SUMMARY OF THE INVENTION

The present application generally relates to systems and methods for creating ablation zones in human tissue. More specifically, the present application relates to the treatment of atrial fibrillation of the heart using ultrasound energy. While the present application emphasizes treatment of atrial fibrillation, one of skill in the art will appreciate that this it not intended to be limiting, and that the systems and methods disclosed herein may also be used to treat other arrhythmias such as ventricular fibrillation, as well as other tissues and conditions.

In a first aspect of the present invention, a method for echo-anatomically mapping tissue comprises advancing a catheter toward a target treatment tissue. The catheter comprises a proximal end, a distal end, an ultrasound transducer adjacent the distal end, and a console adjacent the proximal end. The console is configured to control movement of the catheter, and the ultrasound transducer is configured to sense the target treatment tissue. A first region of the target treatment tissue is sensed with the ultrasound transducer while moving the ultrasound transducer along a first sensing pattern. A first 3-dimensional surface map of the first region is generated. A second region of the target treatment tissue is sensed with the ultrasound transducer while moving the ultrasound transducer along a second sensing pattern. A second 3-dimensional surface map of the second region is generated. The first and the second 3-dimensional surface maps are combined to form a combined surface map.

The advancing step may comprise percutaneously introducing the catheter into vasculature of a patient and transseptally passing the catheter through an atrial septal wall of the patient's heart into a left atrium. Sensing of the first or the second region may comprise operating the transducer in amplitude mode (A-mode). The first or the second sensing pattern may comprise a raster pattern or a spiral pattern. Sensing of the first or the second regions may also comprise delivering a beam of ultrasound energy from the transducer to the target treatment tissue. The sensing of the first or the second regions may be performed without establishing direct contact between the transducer and the tissue. The first sensed region may be the same or different than the second sensed region. The first sensing pattern may be the same or different than the second sensing pattern.

Generating the first or the second 3-dimensional surface map may comprise visually displaying the combined surface map.

The method may further comprise identifying anatomical features in the first sensed region or the second sensed region. The anatomical features in the first or the second region may comprise one or more pulmonary veins. The identifying step may comprise capturing data indicating distance between the transducer and the target treatment tissue at a plurality of points along the first or the second sensing patterns.

The method may also comprise ablating the target treatment tissue with the ultrasound transducer while moving the ultrasound transducer along a first ablation path. The first ablation path may form a lesion around the identified anatomical features. The lesion may block aberrant electrical pathways in the tissue so as to reduce or eliminate atrial fibrillation. The ablating step may comprise selecting the first ablation path from a catalog of available lesion paths based on the identified anatomical features. The first ablation path may be automatically selected from the catalog of available lesion paths, or a physician may prescribe the first ablation path. The method may further comprise accepting or rejecting the selected ablation path by a physician or other operator. A physician or other operator may also modify the selected ablation path. The catalog of available lesion paths may be stored on a memory element coupled to the console. The method may further comprise adding, deleting, or modifying lesion paths stored on the memory element. The ablating may be performed without establishing direct contact between the transducer and the tissue. The method may comprise drawing the first ablation path by a physician or other operator, or the first ablation path may be suggested by the console.

The method may further comprise visually displaying the combined surface map. The method may also comprise superimposing the first ablation path on the combined surface map, and the resulting superimposed map may be visually displayed. The method may further comprise monitoring deviations from the selected lesion path during the ablating. The ablating may be corrected so as to minimize deviations from the selected lesion path. The correction may comprise moving the transducer. Sensing of the first or the second region may also be synchronized with a patient's the cardiac cycle. The method may further comprise determining lesion thickness along the first ablation path.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 schematically illustrates an exemplary catheter system for ultrasonically sensing and ablating cardiac tissue.

FIGS. 2A-2D illustrate exemplary sensing patterns.

FIGS. 3A-3F illustrate exemplary 3D maps for six neighboring portions of the atrial tissue surface.

FIG. 3G illustrates a 3D map obtained by combining the six maps of FIGS. 3A-3F.

FIGS. 4A-4J illustrate exemplary lesion paths.

FIGS. 5A-5B illustrate exemplary ablation lesions.

### DETAILED DESCRIPTION OF THE INVENTION

Overview. The present disclosure emphasizes, but is not limited to catheter systems and methods for ultrasonically sensing and ablating tissue to treat atrial fibrillation. A catheter equipped with an ultrasonic transducer is used to sense and scan at least some portion of atrial heart tissue surface. The ultrasonically sensed data is then used to generate a 3-dimensional (3D) echo-anatomical map of the tissue surface. One or more anatomical features are then identified based on the generated 3-dimensional map. The anatomical features may then be electrically isolated using tissue ablation. In one embodiment, the anatomical features are pulmonary veins (PVs).

Once the anatomical features are identified, a lesion path is chosen so as to surround the anatomical features. In one embodiment, the lesion path is chosen from among a catalog of available lesion paths, based on the location of the identified anatomical features. Alternatively, a physician may prescribe the lesion path by drawing the lesion path around identified features. Once the lesion path is chosen, the catheter is used to ultrasonically ablate the tissue along the lesion path and around the identified anatomical features.

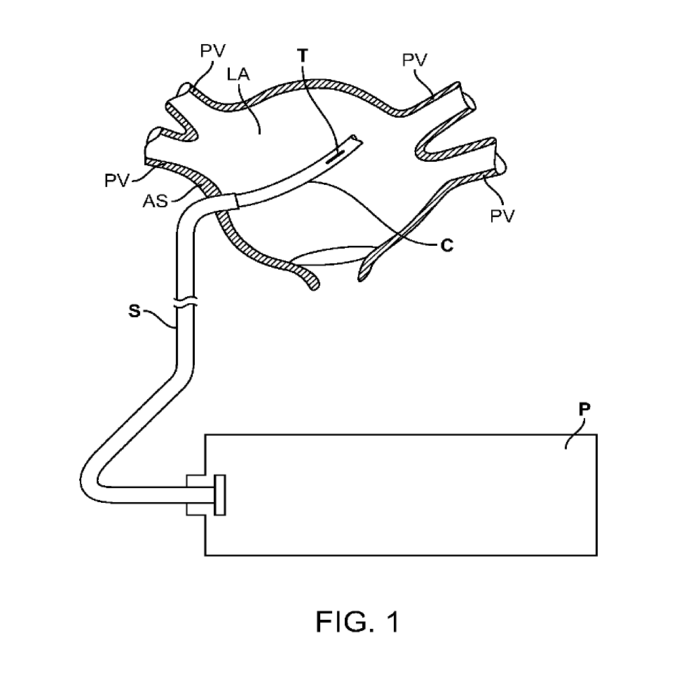

Sensing and ablation system. FIG. 1 is a diagrammatic illustration of an exemplary catheter system for ultrasonically sensing and ablating tissue to treat atrial fibrillation, according to one embodiment of the present invention. The system comprises a sensing and ablation catheter C transseptally disposed across an atrial septal wall AS into the left atrium LA of a patient's heart adjacent the pulmonary veins PV, and a console P outside of the patient. Catheter C comprises a proximal portion and a distal portion. The distal portion of the catheter C is configured for introduction into an atrium of the heart, either percutaneously or surgically, and comprises an ultrasonic transducer subassembly T (hereinafter also referred to as transducer T).

The transducer T is capable of ultrasonically sensing tissue, as well as ultrasonically ablating tissue, without necessarily establishing direct physical contact with tissue. The distal portion of the catheter C is configured to be moveable in a controlled fashion so that it may trace out sensing patterns and lesion paths. In one embodiment, and as shown in FIG. 1, the catheter device C is housed within a sheath S.

The console P is configured to couple to the proximal portion of the catheter C in order to direct the distal tip of catheter C to move in one or more directions, thereby guiding the transducer T along one or more sensing patterns or lesion paths. The console P also controls the operation of transducer T by delivering electrical energy to the transducer T in order to generate ultrasonic energy for sensing and ablating tissue, and by recording scan signals produced by transducer T as it senses the tissue surface.

As mentioned above, the console P controls the catheter C to move in a pattern, such as a raster pattern, in order to scan some portion of the tissue. Based on the received scan signals, console P then generates a 3-dimensional map of the tissue portion.

Based on the 3-dimensional map of the tissue portion, the console P presents one or more anatomical features, such as PVs, that are to be electrically isolated. The console P then suggests a lesion path based on the map and the location of the anatomical features, or a physician may select or prescribe the lesion path. Upon confirmation or modification of the lesion path by a user, the console P directs the catheter C to ablate the tissue along the lesion path.

In one embodiment, console P houses, or is coupled to, a memory element that stores a catalog of available lesion paths, from which catalog the lesion path is selected. The catalog may be configurable, and lesion paths may be added, deleted or modified. In one embodiment, the system further comprises a computer display or monitor in order to present the tissue map, the identified anatomical features, and the suggested lesion path to the user.

Additional details about the catheter C, transducer T, console P, and sheath S are disclosed in U.S. Provisional Patent Application No. 61/254,997 (Attorney Docket No. 027680-001900US), previously incorporated by reference. Other disclosure applicable to the ablation system described above is included in patent applications previously incorporated herein by reference.

Sensing mode. In operation, the transducer T functions in one of two modes: a sensing mode and an ablation mode. When operating in sensing mode, the transducer T is directed to move in a sensing pattern over a portion of atrial tissue surface, and to capture a set of ultrasonically generated data indicating the distance between the transducer T and the atrial tissue at a plurality of points along the traversed sensing pattern. In one embodiment, transducer T operates in Amplitude-mode (A-mode) to sense a distance between the transducer T and the tissue surface.

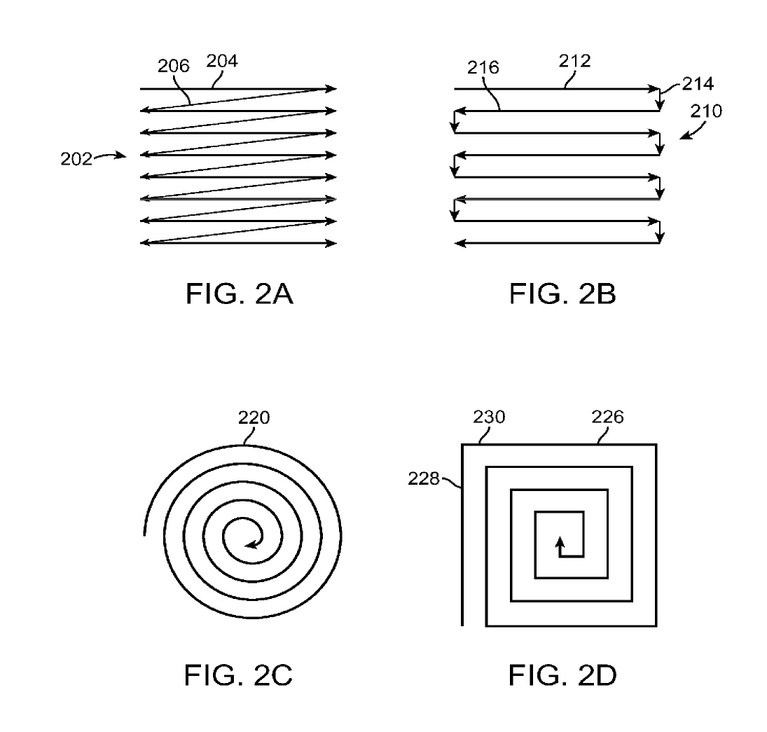

The sensing pattern may be a raster pattern, as shown in the examples of FIGS. 2A-2B. FIG. 2A illustrates a raster pattern 202 where the raster pattern scans horizontally from left to right 204 and a diagonal return 206 from right to left allows the next horizontal scan to begin again from left to right and the pattern is repeated multiple times. FIG. 2B illustrates a variation of a raster scan 210 in which scanning occurs horizontally 212 from left to right. At the end of the horizontal scan, the scan is vertically moved downward as indicated by arrow 214, and then the scan continues from right to left 216. Another vertical adjustment moves the scan downward again, and then the scan from left to right begins again. This pattern is repeated multiple times. FIGS. 2C-2D illustrate exemplary scan patterns having spiral shapes. For example, in FIG. 2C, the scan pattern has a curved pattern that spirals centrally inward to a central point, with each spiral having a smaller radius than the previous spiral. FIG. 2Dillustrates a square spiral, where the scan pattern 226 has a series of vertical 228 and horizontal 230 scans that are joined together to form an inwardly directed square spiral. One of skill in the art will appreciate that the directions left, right, vertical and horizontal may be changed, and therefore are not intended to be limiting. The sensed data is then used by the console P to generate a 3-dimensional surface map of the sensed portion of the atrial tissue. Thus the present system is useful for echo-anatomical mapping of the target tissue surface, such as a portion of, or the entire surface of the left or right atrium of the heart. The surface map may include the entire target treatment surface, or it may include only a section of the treatment surface. Because the catheter may require repositioning several times during mapping of the entire surface, it may be easier to map a section of the target surface, reposition the catheter, and then map another section. Also, in addition to positioning requirements, scanned sections may be limited to certain areas due to memory or data processing limitations of the system.

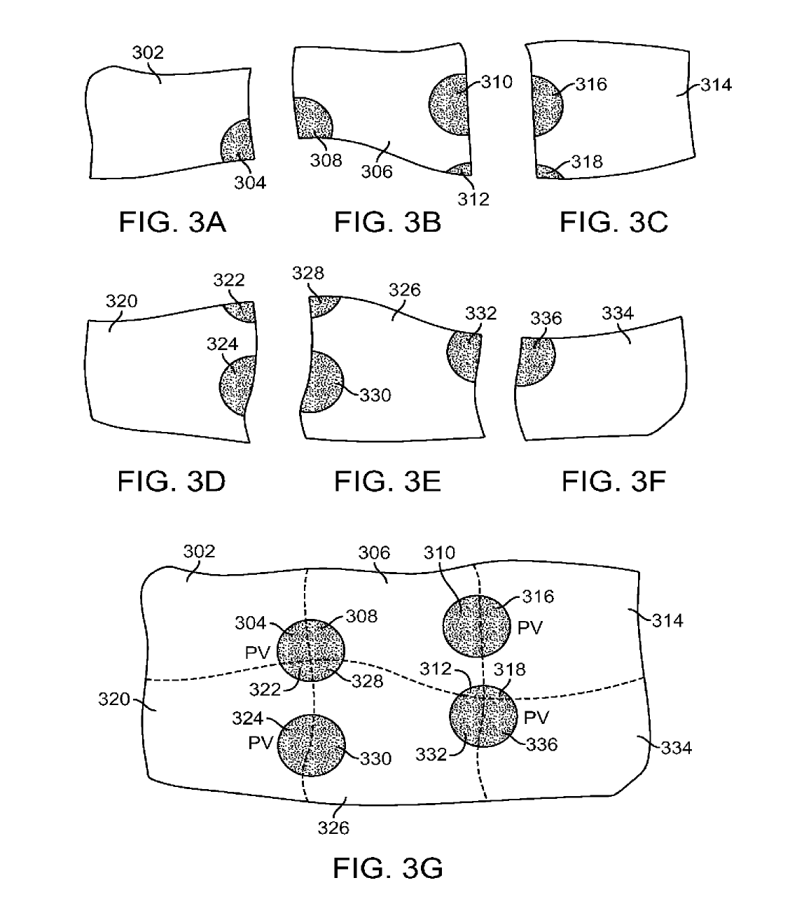

This process of sensing and obtaining scan signals is repeated as needed in order to generate one or more further 3-dimensional maps for one or more neighboring portions of the atrial tissue surface, thereby covering the surface area that is to be mapped with sensing patterns. As one example, FIGS. 3A-3F show 3-dimensional maps for six neighboring portions of the atrial tissue surface having four pulmonary veins PV. FIG. 3A illustrates an upper left portion 302 of the target tissue and shows an upper left portion 304 of a first PV. FIG. 3B illustrates an upper center portion 306 of the target tissue and shows an upper right portion 308 of the first PV, an upper left portion 3 lOof a second PV and an upper left portion 312 of a third PV. FIG. 3C illustrates an upper right portion 314 of the target tissue and shows an upper right portion 316 of the second PV and an upper right portion 318 of the third PV. FIG. 3D illustrates a lower left portion 320 of the target tissue and shows a lower left portion 322 of the first PV and a lower left portion 324 of a fourth PV. FIG. 3E illustrates a lower center portion 326 of the target tissue and shows a lower right portion 328 of the first PV, a lower right portion 330 of the fourth PV and a lower left portion 332 of the third PV. FIG. 3F illustrates a lower right portion 334 of the target tissue and shows a lower right portion 336 of the third PV. The PVs are depicted as grey portions, indicating “holes” or regions of large distance between the transducer T and tissue. Once generated, these one or more 3-dimensional maps may be combined by the console P to form a combined 3-dimensional map of the scanned atrial tissue surface. FIG. 3G shows an exemplary combined 3-dimensional map obtained by combining the six maps of FIGS. 3A-3F. Thus, the present system is capable of mapping a portion of, or mapping the entire inner surface of an atrium, or other tissue surface. Note that in some applications of the present invention it may be determined that obtaining a single 3-dimensional map may be sufficient to allow identification of one or more PVs (instead of obtaining and combining a plurality of 3-dimensional maps, as described above). In the following description, the term “combined map” shall also refer to such a single 3-dimensional real time echo-anatomical map obtained in such embodiments. In preferred embodiments, the map is also compatible with other mapping and ablation systems, such as the CARTO® electroanatomical mapping system (Biosense Webster, Diamond Bar, Calif), CT scanning systems, and the EnSite Array™ from St. Jude Medical, or other similar systems.

The combined echo-anatomical map is then used to identify the location of one or more PVs, which may appear as holes or similar artifacts on the map. The identification of the PV locations may be done algorithmically by the console P, or it may be done by a human user, or by using a combination of user input and programmed logic. Optionally, the echo-anatomical map may be presented to a user on a computer display in order to allow visual identification and/or visual verification of the PV locations.

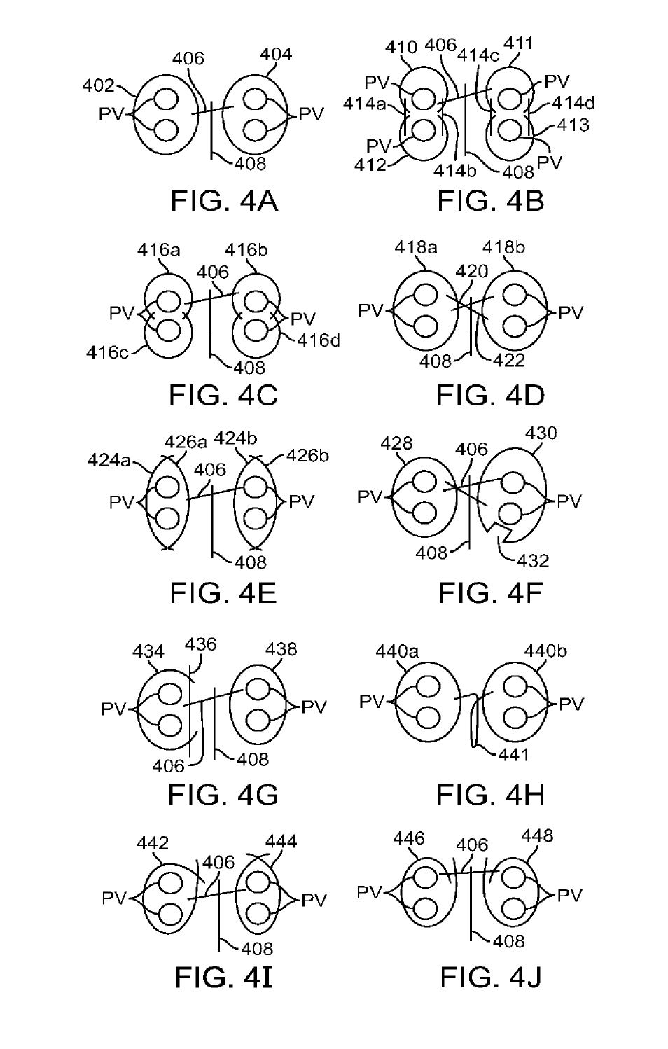

In one embodiment, once the PVs are located, a lesion path is selected from among the catalog of available lesion paths. FIGS. 4A-4J show example lesion paths in a catalog of lesion paths. FIG. 4A illustrates oval or circular lesions 402, 404 encircling two pulmonary veins each (e.g. two left pulmonary veins and two right pulmonary veins). A linear lesion 406 connects each of the oval lesions 402, 404 and a transverse lesion 408 extends from the linear lesion 406 downward toward the mitral valve (not illustrated). FIG. 4B illustrates another embodiment where an arcuate lesion 410 preferably U-shaped, or horseshoe shaped, is superior to, and partially encircles a first upper PV, and a second arcuate lesion 411, that may take the same form as lesion 410 is superior to, and partially encircles a second upper PV. An arcuate lesion 412, preferably U-shaped, or horseshoe-shaped is inferior to, and partially encircles a third PV, and another arcutate lesion 413 that may take the same form as lesion 412 is inferior to, and partially encircles a fourth PV. In this exemplary embodiment, the first PV is superior to the third PV and the fourth PV is inferior to the second PV. Also, in this exemplary embodiment, the first and third PVs are disposed to the left of the second and fourth PVs. Thus, some of the PVs may be left pulmonary veins, and some of the PVs may be right pulmonary veins. Linear lesions 414a, 414b connect the superior arcuate lesion 410 with the inferior lesion 412 so that the first and third PVs are completely encircled. Linear lesions 414c, 414d connect the superior arcuate lesion 411 with the inferior lesion 413 so that the second and fourth PVs are completely encircled. Linear lesion 406 connects the lesions encircling the pairs of PVs and a transverse lesion 408 extends from the linear lesion 406 downward toward the mitral valve (not illustrated). FIG. 4C illustrates still another embodiment where lesions 416a, 416b, 416c, and 416d arc around each of four PVs, such that two pairs of arcs 416a, 416c, and 416b, 416d merge together such that each pair completely encircles two PVs. A horizontal lesion 406 and a transverse lesion 408 connect the lesions encircling two PVs. FIG. 4D illustrates yet another embodiment of a lesion pattern where two oval or circular lesions 418a, 418b each completely encircle two PVs. Two linear lesions 420, 422 join the two oval lesions 418a, 418b forming an “X.” A transverse lesion 408 extends from the “X” downward toward the mitral valve (not shown). FIG. 4E illustrates another embodiment of lesion where two arcs 424a, 426a completely encircle two PVs. The first arc 424a partially encircles one side of the pair of PVs, and the second arc 426a partially encircles the opposite side of the pair of PVs. The ends of the two arcs 424a, 426 acrossover or intersect with one another to form a closed loop. Similarly, another pair of arcs 424b, 426 bcompletely encircle a second pair of PVs. The third arc 424b partially encircles one side of the second pair of PVs, and the fourth arc 426b partially encircles the opposite side of the second pair of PVs. The ends of the third and fourth arcs 424b, 426b crossover one another or intersect with one another to form a closed loop. The pattern also includes a linear lesion 406 and a transverse lesion 408 that generally take the same form as previously described. FIG. 4F illustrates another lesion pattern having an oval or circular lesion 428 encircling two PVs. A second oval or circular lesion 430 encircles another two PVs, and also has a square or rectangular notch 432 to exclude the notched region from being encircled by the lesion. A linear lesion 406 connects the two lesions 428, 430 and a transverse lesion 408 extends therefrom. FIG. 4G shows another exemplary lesion pattern with an arc 434 partially encircling two PVs and a linear lesion 436 crossing both ends of the arc 434 so that the resulting lesion completely encircles both PVs. A second oval or circular lesion 438 completely encircles two other PVs and a linear lesion 408 connects the two lesions 436, 438. A transverse lesion 408 extends from the linear lesion 406. FIG. 4H shows another exemplary lesion having a curved lesion 441 connecting two circular or oval lesions 440a, 440b each encircling two PVs. The curved lesion 441 has two ends that overlap with each of the oval lesions 440a, 440b, and the curved lesion also overlaps itself, forming a lower loop similar to the Greek letter gamma. FIG. 4 lillustrates a first loop 442 that encircles two PVs, and a second loop 444 that encircles two additional PVs. Each loop has overlapping ends such that the two PVs are completely encircled. A linear lesion 406 connects the two loops 442, 444 and a transverse lesion 408 extends from the linear lesion 406. FIG. 4J shows another embodiment where loops 446, 448 encircle two PVs each. However, in this embodiment, the ends of the loops do not overlap with one another and thus, while the PVs are completely encircled, a total conduction block has not been created, as the aberrant electrically activity can pass between the ends of the loops which do not overlap. Therefore, a linear lesion 406 extends through the open portions of each loop, and between both loops 446, 448, creating the conduction block. A transverse lesion 408 extends from the linear lesion 406.

The catalog of ablation patterns may be stored on a memory element coupled to the console P, or otherwise be made accessible to the console P. The choice of the particular lesion path to be used for ablation is based on the identified locations of the PVs in the combined 3-dimensional map of the atrial tissue, with the lesion path chosen to surround the PVs in order to electrically isolate them and thereby treat atrial fibrillation.

In one embodiment, the console P may be programmed to suggest a lesion path based on image analysis techniques applied to the obtained tissue map in order to locate artifacts, such as holes or ovals, which indicate the location of PVs. The user (for example, a surgeon) may then accept the suggested lesion path, modify the suggested lesion path, choose another lesion path from the catalog, or draw a new lesion path. In such an embodiment, the console P may superimpose the selected lesion path onto the obtained surface map and present them to the user, thereby allowing the user to make any needed modifications prior to ablation.

Additionally and optionally, the console P may be configured to learn from the user's (i.e., surgeon's) input with respect to lesion choices and lesion path modifications, by storing such information and associating it with the corresponding tissue maps and identified PV locations, for future reference. This allows the console P to personalize lesion path choices to particular surgeons, to suggest lesion paths based on past choices aggregated over a number of surgeons, etc.

Additional details on sensing and mapping may be found in U.S. patent application Ser. No. 12/609,759 (Attorney Docket No. 027680-001110US); Ser. No. 12/609,274 (Attorney Docket No. 027680-001410US); and Ser. No. 12/609,705 (Attorney Docket No. 027680-001610US), each previously incorporated herein by reference. Other details which may be applicable are disclosed in other patent applications previously incorporated herein by references.

Ablation mode. Once a lesion path is chosen, the console P causes the transducer T to switch to operating in ablation mode. In ablation mode, the electrical energy delivered to the transducer T, and therefore the ultrasonic energy delivered by the transducer T to the tissue, is higher than in sensing mode, and sufficient to ablate the tissue. In this mode, the console P directs the catheter C to move the transducer T along the chosen lesion path while the transducer T ultrasonically ablates atrial tissue along the chosen lesion path, thereby creating an ablation lesion around the one or more PVs.

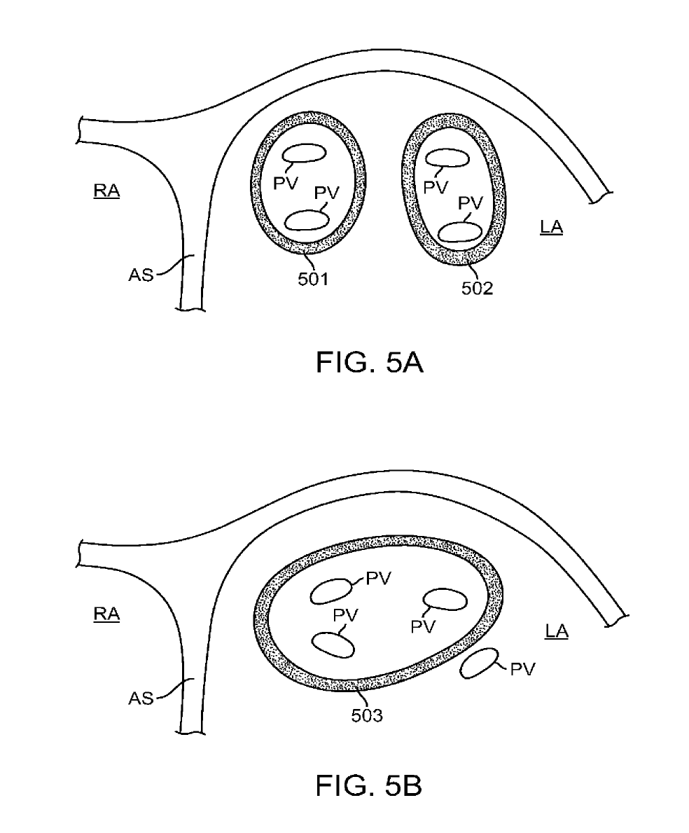

FIGS. 5A and 5B show example lesions created in the left atrium LA of the heart. In this embodiment, the left atrium LA has four pulmonary veins PV. The left atrium is separated from the right atrium via an atrial septal wall AS. FIG. 5A shows an exemplary lesion 501 created around two PVs and lesion 502 created around another two PVs. Both lesions 501, 502 may be circular, elliptical, oval, or another shape (e.g. square, rectangular, etc.) and completely encircle two PVs. FIG. 5B shows an exemplary lesion 503 created around three PVs in the left atrium LA. The lesion 503 may be circular, oval, elliptical, or any other shape (e.g. square, rectangular, etc.) that completely encircles the three PVs. In an optional embodiment, the console P may be configured to monitor deviations from the chosen lesion path and to provide corrections by adjusting the movement of the catheter C. For example, the console P may be configured to monitor a distance between the chosen lesion path and the tissue site that is being ultrasonically ablated by the transducer T, and move the distal portion of the catheter C (and with it the transducer T) towards the chosen lesion path in order to decrease that distance, thereby repositioning the transducer T along the chosen lesion path. In another optional embodiment, the console P may be configured to detect the transducer's T passing over veins and provide a visual indication thereof (for example, by flashing a red light when going over a vein and a green light otherwise), thereby giving an opportunity to the surgeon to intervene or to provide corrections at a later time.

Additionally and optionally, the console P may be configured to synchronize the operation of the transducer T, in sensing mode and/or in ablation mode, with the cardiac cycle. This is to enable greater accuracy in sensing and/or in ablation given the beating of the heart. Such synchronization may be accomplished by configuring the console P to receive input from a monitoring device such as an electrocardiograph (EKG), a computed tomography (CT) scanner, an electroanatomical mapping system (CARTO), or other such devices. The operation of the transducer T is then synchronized to accommodate or better account for the movement of the heart. For example, the console P may synchronize with the cardiac cycle and cause the transducer T to operate within periodic time slices in the cardiac cycle where the movement of the heart tissue is at a minimum, such as during physical diastole when the heart is stationary for the longest period of time during the cardiac cycle.

Additionally and optionally, the console P may be programmed to analyze the scan signals, received from the transducer T in sensing mode, and infer information about the thickness of the produced ablation. For example, this may be accomplished by comparing the times at which various tissue boundaries reflect the ultrasound emitted by the transducer T, and inferring the distance between such tissue boundaries (i.e., the thickness of the tissue between the boundaries). When applied to the two tissue boundaries on each side of an ablated layer, the ablation thickness may be inferred. Such thickness values may be used to more accurately time the exposure of atrial tissue to ultrasonic ablation energy, thereby providing for substantially transmural ablation and electrical isolation of the PVs. Additional details about characterizing the lesion is disclosed in patent applications previously incorporated herein by reference.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for mapping and ablating tissue with a catheter advanced toward a target tissue, the catheter comprising a proximal end, a distal end, and a transducer adjacent the distal end, said method comprising:
sensing a location and a thickness of the target tissue with the transducer while the transducer is in a sensing mode while moving the transducer in a sensing pattern;
generating a 3D map of the tissue;
identifying one or more anatomical features within the 3D map;
drawing a lesion path around the identified one or more anatomical features;
switching the transducer from the sensing mode to an ablation mode; and
ablating the target tissue along the lesion path with ultrasound energy from the transducer by moving the catheter along the lesion path after switching the transducer from the sensing mode to the ablation mode,
wherein the catheter is configured to be coupled to a console adjacent the proximal end, and
wherein the console is configured to control movement of the catheter while ablating the target tissue along the lesion path and to adjust the movement of the catheter based on the sensed thickness to control exposure time of the target tissue to the ultrasound energy while ablating while also adjusting catheter the movement of the catheter to ensure no deviations from the drawn lesion path.

2. The method of claim 1, wherein the catheter is advanced toward the target tissue by being percutaneously introduced into vasculature of a patient and being transeptally passed through an atrial septal wall of the patient's heart into a left atrium.

3. The method of claim 1, wherein the target tissue comprises left atrial tissue.

4. The method of claim 1, wherein the sensing mode comprises amplitude mode (A-mode).

5. The method of claim 1, wherein the sensing pattern comprises a spiral pattern.

6. The method of claim 1, wherein the sensing and ablating are performed without direct contact between the transducer and the target tissue.

7. The method of claim 1, wherein the one or more anatomical features comprise one or more pulmonary veins.

8. The method of claim 1, wherein the identifying step comprises capturing data indicative of a distance between the transducer and the target tissue at a plurality of points along the sensing pattern.

9. The method of claim 1, wherein the lesion path is drawn to completely surround and electrically isolate the one or more anatomical features.

10. The method of claim 1, wherein the lesion path is drawn to partially surrounds the one or more anatomical features.

11. The method of claim 1, further comprising modifying the drawn lesion path after it has been drawn.

12. The method of claim 1, further comprising visually displaying the lesion path superimposed on the 3D map of the tissue.

13. The method of claim 1, wherein the exposure time to the ultrasound energy is controlled by varying a speed of movement of the catheter.

14. The method of claim 1, wherein the sensed thickness is derived by comparing a first time at which a first tissue boundary of the target tissue reflects ultrasound energy emitted by the transducer to a second time at which a second tissue boundary of the target tissue reflects ultrasound energy emitted by the transducer and inferring the distance between the first and second tissue boundaries from on the first and second times.

15. The method of claim 1, wherein the exposure time to the ultrasound energy is adjusted to ensure transmural lesions throughout the lesion path.

16. The method of claim 1, further comprising displaying the 3D map on a computer display, wherein drawing the lesion path comprises drawing the lesion path on the displayed 3D map.

17. The method of claim 1, the console is configured to suggest preferred locations of the lesion path based on image analysis techniques applied to the 3D map in order to facilitate the drawing step.

18. The method of claim 1, further comprising drawing one or more additional lesion paths that intersect with the lesion path.

19. The method of claim 18, wherein the lesion path and the one or more additional lesion paths intersect to electrically isolate the one or more anatomical features.

20. The method of claim 1, wherein the lesion path is drawn to block aberrant electrical pathways in the target tissue so as to reduce or eliminate atrial fibrillation.
